(19) United States
(12) Patent Application Publication
(10) Pub. No.: US 2025/0266169 A1
(43) Pub. Date: Aug. 21, 2025

(54) MODEL BUILDING METHOD, AND MONITORING METHOD AND SYSTEM FOR ANIMAL HEALTH STATUS

(71) Applicant: Petcision Limited, Kowloon (HK)

(72) Inventor: Chi Tung CHOY, Kowloon (HK)

(21) Appl. No.: 18/928,047

(22) Filed: Oct. 27, 2024

(30) Foreign Application Priority Data
Feb. 20, 2024 (HK) 32024087336.6

(51) Int. Cl. G16H 50/30; G16B 30/00; G16B 40/20; G16H 10/60; G16H 50/50

(52) U.S. Cl. CPC G16H 50/30; G16B 30/00; G16B 40/20; G16H 10/60; G16H 50/50

(57) ABSTRACT

The present application relates to the field of health management technologies and the like, in particular to a model building method, and a monitoring method and system for animal health status. The method for building a model of animal health status includes the following steps: collecting biological samples of healthy animals and various diseased animals; performing metagenomic sequencing on microorganisms of the various biological samples collected to obtain macroscopic gene information; performing data analysis using an AI algorithm on the obtained macroscopic gene information, to obtain microbiome NGS data of the macroscopic gene information; and building a model of the microbiome NGS data obtained through analysis, including model data for healthy animals and model data for diseased animals. The method can better predict or evaluate disease risks of animals effectively.

### TECHNICAL FIELD

This application relates to the field of health management technologies and the like, in particular to a model building method, and a monitoring method and system for animal health status.

### BACKGROUND

The field of microbial biotechnology and bioinformatics has seen significant advancements in recent years, particularly with the advent of Next-Generation Sequencing (NGS). Recent scientific studies have already demonstrated that microbiota composition is closely linked to various physiological functions in animals, including nutrient absorption, immune system regulation, and metabolic processes. Therefore, any imbalance in the microbiome can result in a range of health issues, such as obesity, gastrointestinal disorders, and even behavioral problems. The vice versa also holds, in which the microbiome could reflect the health status of cats and dogs. However, there is a growing and unmet need for more efficient and accurate methods for health monitoring in companion animals. Conventional methods take a long time to diagnose and require invasive sampling on-site such as blood collection, our microbiome-based analysis combined with advanced artificial intelligence (AI) analytics provides a truly non-invasive and innovative method that provides timely health management and early intervention windows to improve the health status of companion animals.

### SUMMARY

To achieve better effective disease risk prediction or evaluation for animals, the present application provides a model data building method, and a monitoring method and system for animal health status, which are all based on macroscopic gene information of microorganisms in biological samples of animals, rather than use any one or several microorganisms as indexes. Instead, the macroscopic gene information of microorganisms covered in the biological samples is used as the foundation of sequencing and a data source of model building.

According to the present application, metagenomic sequencing is performed on a certain number of healthy animals and diseased animals, and microbiome NGS data obtained using an AI algorithm is correlated with animal health status, disease status (type and severity) of the data source, to obtain the foundation of model building. By comparing the model with microbial NGS data of a target animal obtained in the same method, the health status, disease, risk and probability of the target animal can be obtained through analysis for corresponding monitoring, evaluation and analysis. After an obtained result is fed back, the animal can be purposefully nursed to reduce the possibility of disease.

Therefore, the present application provides a first scheme, that is, a method for building a model of animal health status, which includes the following steps:

step 101: collecting biological samples of healthy animals and various diseased animals;

step 102: performing metagenomic sequencing on microorganisms of the various biological samples collected in step 101 to obtain macroscopic gene information;

step 103: performing data analysis using an AI algorithm on the macroscopic gene information obtained in step 102, to obtain microbiome NGS data of the macroscopic gene information; and

step 104: building a model of the microbiome NGS data obtained through analysis in step 103, including model data for healthy animals and model data for diseased animals.

Optionally, the biological samples in step 101 are any one of animal stool, animal saliva, animal skin swab, or animal rectal swab.

Optionally, the microorganisms in step 102 include bacterial 16S rRNA, fungal ITS, and virus.

Optionally, the building a model of the data in step 104 includes the following steps:

step 1041: the obtained microbiome NGS data is preprocessed to remove noise and standardize a format, and preprocessed data is divided into a training set and a test set;

step 1042: data of the training set is used to establish a correlational model between data and disease, and data of the test set is used to compare a health status result obtained after the data is input into the model with an actual result, to test measurement metrics of the model; and

step 1043: if the measurement metrics are satisfactory, the model is established; or if the measurement metrics are unsatisfactory, step 1042 is repeated.

Optionally, the measurement metrics in step 1042 include at least one of accuracy, precision, recall, and F1 score.

The present application further provides a second scheme, that is, a method for monitoring animal health status, which includes the following steps:

step 105: collecting biological samples of a target animal;

step 106: performing metagenomic sequencing on microorganisms of the biological samples collected in step 105 to obtain corresponding gene information;

step 107: performing data analysis using an AI algorithm on the gene information obtained in step 106, to obtain microbiome NGS data of the gene information; and

step 108: comparing the microbiome NGS data obtained through analysis in step 107 with the constructed model of claim 1 to evaluate a health status of the animal.

Optionally, the biological samples in step 105 are any one of animal stool, animal saliva, animal skin swab, or animal rectal swab.

Optionally, the microorganisms in step 106 include bacterial 16S rRNA, fungal ITS, and virus.

Optionally, the AI algorithm in step 107 includes at least one of ensemble, boosting, decision tree, support vector, logical or linear regression, and neural network.

The present application further provides a third scheme, that is, a system for monitoring animal health status, where the monitoring system uses the above-described model building method and monitoring method.

The present invention is a novel product for health monitoring of companion animals by utilizing AI and microbiome data generated by NGS. Data collection starts with a stool sample collection kit, which allows a pet owner to collect a stool sample at home without prior knowledge or training. The sample will be subjected to nucleic acid extraction and metagenomic sequencing (bacterial 16S rRNA, fungal ITS and virus). Health risk prediction powered by AI will then be performed based on microbiome profile and dysbiosis state to identify patterns and anomalies that could indicate the risk of the pet bearing certain diseases (including but not limited to diabetes, cancers, periodontal diseases, heart diseases, cognitive function degradation due to aging). The AI algorithm is trained on a vast dataset of microbiome data, allowing it to accurately detect diseases even in complex cases. Key technical terms used to describe the present invention include “artificial intelligence,” “next-generation sequencing,” “bioinformatics,” “microbiome,” “metagenomics,” “health monitoring,” and “disease risk prediction.”

A preferred embodiment of the present invention relates to a method for achieving robust and accurate health risk prediction universally applicable to any type of companion animal. The present invention is built on a powerful AI platform that uses machine learning algorithms (including but not limited to ensemble, boosting, decision tree, support vector, logistic or linear regression, and neural network) for data analysis. The microbiome NGS data generated through metagenomic sequencing (including but not limited to bacterial 16S rRNA, fungal ITS and virus) will be used as input, and the AI algorithm will analyze the data to detect any signs of disease. The construction of the system includes integrating the AI platform with an interface for inputting and outputting NGS data. Principles involved include machine learning, metagenomics, microbiome, and disease pathology. The method includes training the AI algorithms on a dataset of metagenomic sequences, and then using these trained algorithms to analyze new NGS data for disease detection.

### BRIEF DESCRIPTION OF DRAWINGS

FIG. 1 is a flowchart of a building method and monitoring method for animal health status according to the present application;

FIG. 2 is a schematic diagram of alpha diversity, expressed in the number of species, based on Faith's phylogenetic distance (Faith PD) between adult and senior animal subgroups, where green represents healthy and pink represents diseased;

FIG. 3 is a schematic diagram of a principal coordinate analysis (PCoA) biplot based on Bray-Curtis distances between animals, where green triangle represents healthy and pink circle represents diseased;

FIG. 4 is a schematic diagram of a PCoA biplot based on Jaccard distances between animals, where green triangle represents health and pink circle represents diseased;

FIG. 5 is a block diagram of Firmicutes/Bacteroidetes (F/B) ratio between animals, where green represents healthy and pink represents diseased;

FIG. 6 is a Venn diagram of identified unique representative sequences between animals, where green represents healthy and pink represents diseased;

FIG. 7 shows performance in terms of F1 score of machine learning model training based on microbiome data of diseased small animals in a test group, for example, logarithmic regression model, SVM model and random forest model shown in the figure;

FIG. 8 shows performance in terms of precision of machine learning model training based on microbiome data of diseased small animals in a test group, for example, logarithmic regression model, SVM model and random forest model shown in the figure;

FIG. 9 shows performance in terms of recall of machine learning model training based on microbiome data of diseased small animals in a test group, for example, logarithmic regression model, SVM model and random forest model shown in the figure; and

FIG. 10 shows performance of an area underneath a receiver operating characteristic (ROC) curve of machine learning model training based on microbiome data of diseased small animals in a test group, for example, logarithmic regression model, SVM model and random forest model shown in the figure.

### DETAILED DESCRIPTION

In order to make the objectives, technical schemes and advantages of embodiments of the present application clearer, the technical schemes in the embodiments of the present application are clearly and completely described hereinafter with reference to the embodiments of the present application. It is obvious that the described embodiments are only some of the embodiments instead of all the embodiments of the present application. Generally, the components of embodiments of the present application described and illustrated herein may be arranged and designed in a variety of different configurations.

The following presents various model testing results obtained by using the method of the present application and microbiome NGS data of a certain number of healthy animals and diseased animals calculated using various AI algorithms that have been adopted in conventional technology. From the following description and drawings, it can be learned that there are distinct differences in microbiome NGS data obtained for healthy animals and diseased animals, and such distinct differences are the foundation and core of the model building of data in the present application. After such a model is built, the obtained model can be used to better predict and evaluate the health status and disease risk of a target animal. As can be learned from the following embodiments, the model building method of the present application achieves better results in both precision and recall in a testing process.

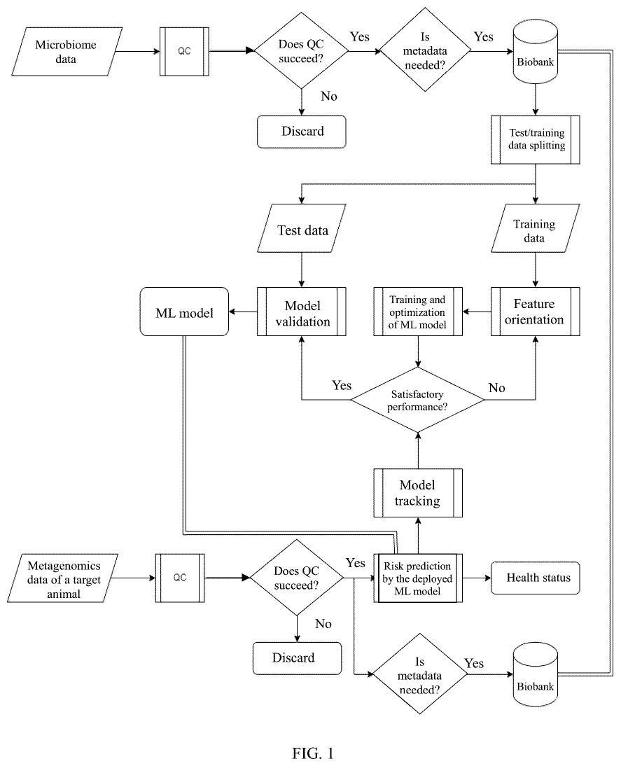

FIG. 1 is a flowchart of a building method and monitoring method for animal health status according to the present application. As shown in FIG. 1, the process begins with collecting data from various sources. These data are then preprocessed to remove any noise and standardize the format. The preprocessed data is then split into a training set and a test set. The training set is used to train an AI model. This includes inputting data into the model and enabling it to learn patterns and relationships of diseases. The training process may involve multiple iterations to optimize the performance of the model. Once the model is trained, the model is tested against the test set by inputting test data into the model and comparing the model's predictions with actual results. Performance of the model will be evaluated by various metrics, including but not limited to accuracy, precision, recall and F1 score. If the performance of the model is satisfactory, the model can be used for disease prediction. If not, the model or training process can be adjusted and the process is repeated until satisfactory performance is achieved.

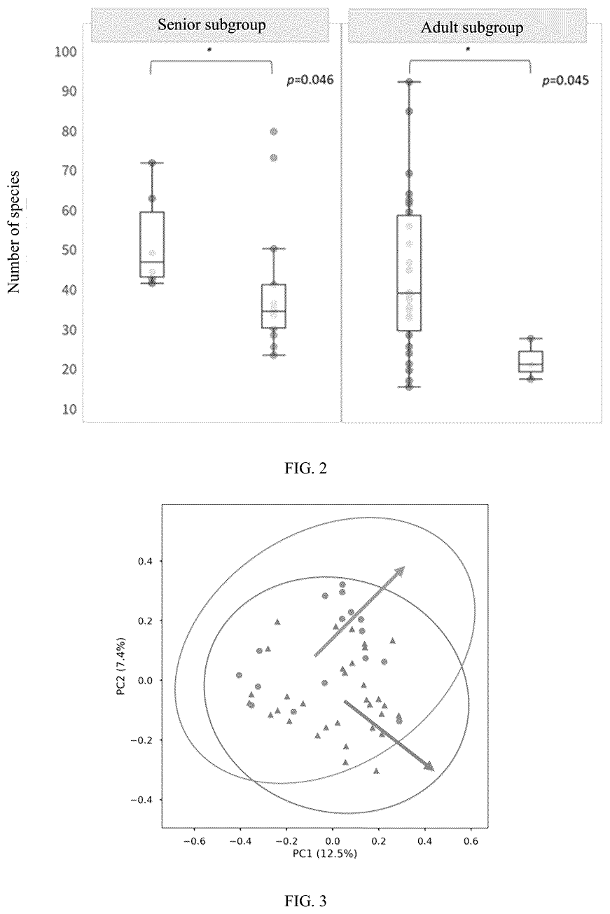

FIG. 2 is a schematic diagram of alpha diversity, expressed in the number of species, based on Faith PD between adult and senior animal subgroups, where green represents healthy and pink represents diseased. As shown in FIG. 2, the boxplot provides a visual representation of the distribution of alpha diversity based on Faith PD between the adult and senior subgroups in healthy and diseased groups. By comparing these boxplots, we can observe differences in alpha diversity between the adult and senior subgroups, as well as between the healthy and diseased people. Alpha diversity refers to species diversity in a specific region or ecosystem, which is usually expressed by the number of species, that is, species richness. This provides a foundation for the AI model to build into health status on microbial diversity and evolutionary diversity.

FIG. 3 is a schematic diagram of a PCoA biplot based on Bray-Curtis distances between animals, where green triangle represents healthy and pink circle represents diseased. As shown in FIG. 3, the PCoA biplot based on Bray-Curtis distances provides a visual representation of differences between microbiomes of healthy and diseased groups. In this biplot, each point represents a sample from either the healthy group (represented by green triangle) or the diseased group (represented by pink circle). The position of each point on the plot is determined by the Bray-Curtis distance between that sample and another sample. This distance is a measure of the difference between the components of two samples. By examining these plots, it is indicated that healthy and diseased small animals have different gut microbiome profiles, which provides a foundation for the AI model to build into health status.

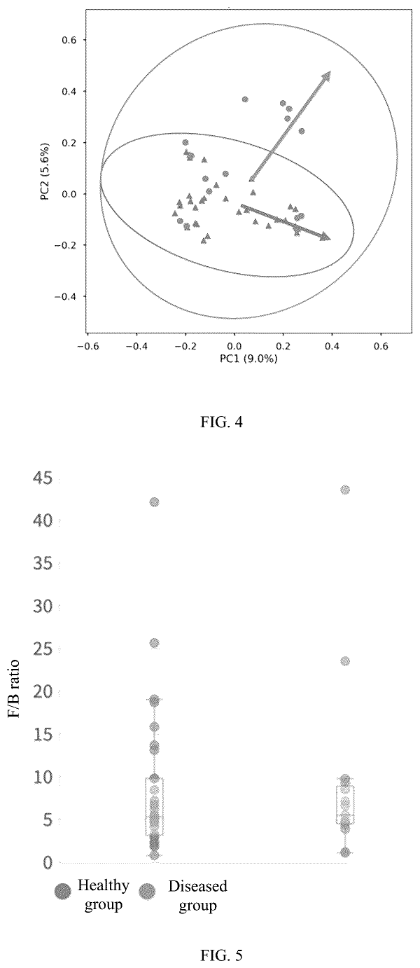

FIG. 4 is a schematic diagram of a PCoA biplot based on Jaccard distances between animals, where green triangle represents healthy and pink circle represents diseased. As shown in FIG. 4, the PCoA biplot based on Bray-Curtis distances provides a visual representation of differences between microbiomes of healthy and diseased groups. In this biplot, each point represents a sample from either the healthy group (represented by green triangle) or the diseased group (represented by pink circle). The position of each point on the plot is determined by the Bray-Curtis distance between that sample and another sample. This distance is a measure of the difference between the components of two samples. By examining these plots, it is indicated that healthy and diseased small animals have different gut microbiome profiles, which provides a foundation for the AI model to build into health status.

FIG. 5 is a block diagram of Firmicutes/Bacteroidetes (F/B) ratio between animals, where green represents healthy and pink represents diseased. As shown in FIG. 5, the block diagram provides a visual representation of the distribution of the F/B ratio distribution between healthy and diseased small animals. A significant difference in the F/B ratio indicates a significant difference in gut microbial composition at a phylum level between the healthy and diseased small animals. This provides a foundation for the AI model to build into health status on microbiome profile.

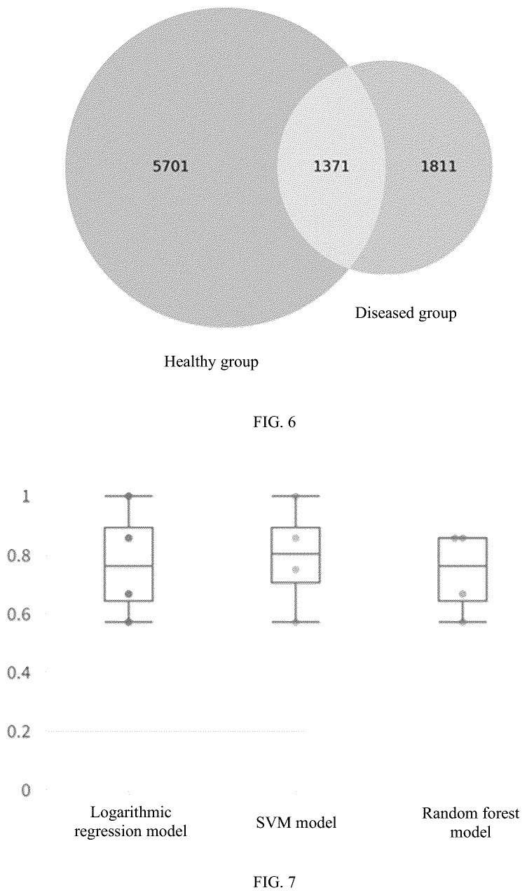

FIG. 6 is a Venn diagram of identified unique representative sequences between animals, where green represents healthy and pink represents diseased. As shown in FIG. 6, the Venn diagram provides a visual representation of the overlap of unique representative sequences (quality-controlled readout of NGS data obtained through metagenomic sequencing) between microbiomes of healthy and diseased groups. Each circle represents a group (healthy or diseased), and an overlapping area between circles represents a shared representative sequence of the two groups. A huge unique area indicates that each group has many representative sequences that are not found in another group, and that their microbiomes are highly different. This provides a foundation for building an AI model on this modality.

FIG. 7 shows performance in terms of F1 score of machine learning model training based on microbiome data of diseased small animals in a test group, for example, logarithmic regression model, SVM model and random forest model shown in the figure. In FIG. 7, the F1 score is a measure of the accuracy of a model on a dataset, combining the precision and recall of the model. Regardless of the model chosen, the block diagram reflects that the model performed better than random guessing with all F1 scores above 0.5 in any iteration. A median F1 score can reach 0.8, which means that the training accuracy of this model is 80%.

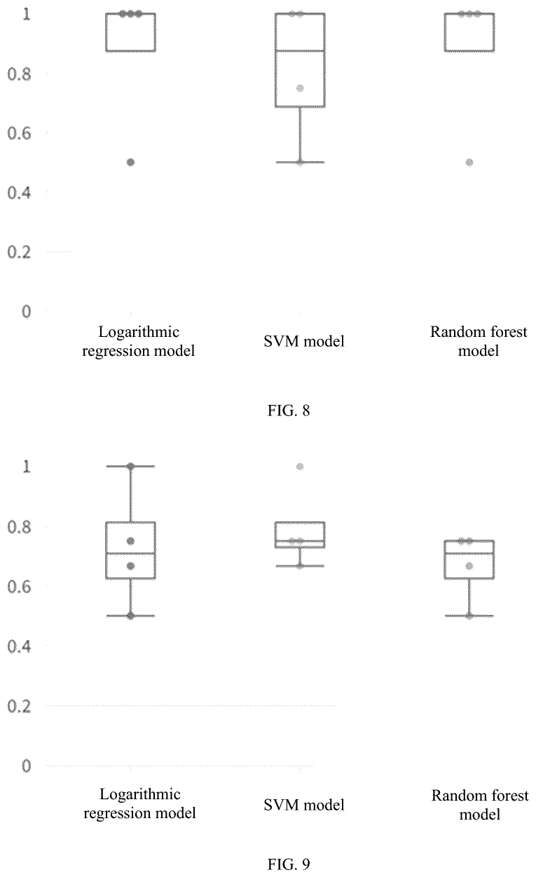

FIG. 8 shows performance in terms of precision of machine learning model training based on microbiome data of diseased small animals in a test group, for example, logarithmic regression model, SVM model and random forest model shown in the figure. As shown in FIG. 8, precision in machine learning refers to an ability of a model to correctly identify labels or classes. It is defined as a ratio of correctly classified positive samples (true positive) to a total number of classified positive samples (correct or incorrect). Median precision may reach 0.9, which means that 90% of high disease risk predictions are correct.

FIG. 9 shows performance in terms of recall of machine learning model training based on microbiome data of diseased small animals in a test group, for example, logarithmic regression model, SVM model and random forest model shown in the figure. As shown in FIG. 9, recall, also known as a true positive rate (TPR), is a percentage of data samples that a machine learning model correctly identifies as belonging to a class of interest out of total samples of the class. It is defined as a ratio of true positives to a sum of true positives and false negatives. Recall measures an ability of the model to find all positive samples. A median recall rate is about 0.7, which means that the model can identify a pattern of 70% of diseased cases microbiome.

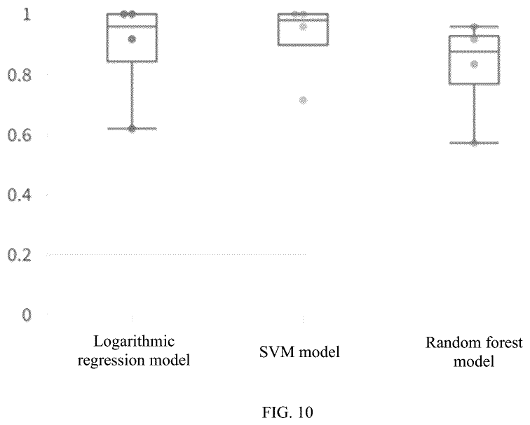

FIG. 10 shows performance of an area underneath a ROC curve of machine learning model training based on microbiome data of diseased small animals in a test group, for example, logarithmic regression model, SVM model and random forest model shown in the figure. As shown in FIG. 10, an area under a (ROC) curve measures an entire two-dimensional area underneath the entire ROC curve. The ROC curve is a graph showing an aggregate measure of performance of all possible classification thresholds, with AUROC of 1 representing a perfect classifier. A median AUROC can reach 0.9, which means that the trained AI model performs well in identifying patterns in microbiome data of diseased small animals.

The above is only the description of some preferable embodiments of the present application, and is not intended to limit the present application. It will be apparent to those of ordinary skill in the art that various modifications and variations can be made to the present application. Any modifications, equivalent substitutions, improvements, etc. made within the spirit and principle of the present application shall fall within the scope of protection of the present application.

1. A method for building a model of animal health status, comprising the following steps:
step 101: collecting biological samples of healthy animals and various diseased animals;
step 102: performing metagenomic sequencing on microorganisms of the various biological samples collected in step 101 to obtain macroscopic gene information;
step 103: performing data analysis using an AI algorithm on the macroscopic gene information obtained in step 102, to obtain microbiome NGS data of the macroscopic gene information; and
step 104: building a model of the microbiome NGS data obtained through analysis in step 103, comprising model data for healthy animals and model data for diseased animals.

2. The model building method of claim 1, wherein the biological samples in step 101 are any one of animal stool, animal saliva, animal skin swab, or animal rectal swab.

3. The model building method of claim 1, wherein the microorganisms in step 102 comprise bacterial 16S rRNA, fungal ITS, and virus.

4. The model building method of claim 1, wherein the AI algorithm in step 103 comprises at least one of ensemble, boosting, decision tree, support vector, logical or linear regression, and neural network.

5. The model building method of claim 1, wherein the building a model of the data in step 104 comprises the following steps:
step 1041: the obtained microbiome NGS data is preprocessed to remove noise and standardize a format, and preprocessed data is divided into a training set and a test set;
step 1042: data of the training set is used to establish a correlational model between data and disease, and data of the test set is used to compare a health status result obtained after the data is input into the model with an actual result, to test measurement metrics of the model; and
step 1043: if the measurement metrics are satisfactory, the model is established; or if the measurement metrics are unsatisfactory, step 1042 is repeated.

6. The model building method of claim 5, wherein the measurement metrics in step 1042 comprise at least one of accuracy, precision, recall, and F1 score.

7. A method for monitoring animal health status, comprising the following steps:
step 105: collecting biological samples of a target animal;
step 106: performing metagenomic sequencing on microorganisms of the biological samples collected in step 105 to obtain corresponding gene information;
step 107: performing data analysis using an AI algorithm on the gene information obtained in step 106, to obtain microbiome NGS data of the gene information; and
step 108: comparing the microbiome NGS data obtained through analysis in step 107 with the constructed model of claim 1 to evaluate a health status of the animal.

8. The monitoring method of claim 7, wherein the biological samples in step 105 are any one of animal stool, animal saliva, animal skin swab, or animal rectal swab.

9. The monitoring method of claim 7, wherein the microorganisms in step 106 comprise bacterial 16S rRNA, fungal ITS, and virus.

10. The monitoring method of claim 7, wherein the AI algorithm in step 107 comprises at least one of ensemble, boosting, decision tree, support vector, logical or linear regression, and neural network.
